(19) United States
(12) Patent Application Publication
(10) Pub. No.: US 2009/0275015 A1
(43) Pub. Date: Nov. 5, 2009

(54) NON-INVASIVE RESPIRATORY RAPID DIAGNOSIS METHOD

(76) Inventor: Aleta Behrman Bonner, Betton, TX (US)

Correspondence Address: David Allen Hall, P.O Box 1540, Blanco, TX, 78606, US

(21) Appl. No.: 12/433,890

(22) Filed: Apr. 30, 2009

Related U.S. Application Data
(60) Provisional application No. 61/126,183, filed on Apr. 30, 2008.

(51) Int. Cl. C12Q 1/70

(52) U.S. Cl. 435/5

(57) ABSTRACT

A non-invasive method for rapidly determining the specific presence or absence of respiratory pathogens and chemical entities.

This application claims priority from and incorporates by reference the provisional patent application 61/126,183 filed by the same sole inventor on Apr. 30, 2008.

### BACKGROUND OF THE INVENTION

Respiratory specimens that are easily obtained for medical, diagnostic, and research purposes consist of secretions from the nose, nasopharynx, oral cavity, pharynx, and oropharynx. These include secretions such as nasal mucus, droplets from coughing or sneezing, saliva, and pharyngeal and oropharyngeal fluid, all of which play an important role in the spread of many types of viral and bacterial pathogens. Infectious diseases caused by these pathogens are commonly spread through the population via direct contact with respiratory secretions, respiratory droplets, and contact with fomites.

Respiratory specimens from acutely ill patients are commonly collected for identification of causative agents for conditions such as Group A Strep pharyngitis and respiratory tract infection due to influenza virus, respiratory syncytial virus (RSV), parainfluenza virus, and adenovirus, among others. Respiratory secretions can also be used to detect illicit drugs, monitor therapeutic drug levels, and diagnose non-infectious conditions such as allergic rhinitis, cystic fibrosis, dental caries, and certain cancers, to name a few.

From a public health perspective, the proper collection and testing of respiratory specimens may play the greatest role in global disease surveillance and detection of immediate threats to the population. Annual influenza surveillance has been conducted for decades in order to determine the vaccine strains that should be used for the next season's influenza vaccine. Disease surveillance has been highlighted as an emergent global issue due to outbreaks of SARS, MRSA, and swine and avian influenza, thus making ongoing surveillance and rapid diagnosis efforts a significant medical priority.

The present invention may be useful in the veterinary field as well as in the field of human medicine. Rapid diagnosis of animal ailments is important for the well-being of pets, wild animals, and domesticated teed stock. The present invention could assume world wide significance where animal disease approaches pandemic or epidemic levels. The present invention applies to the entire animal kingdom.

Critical to the success of respiratory specimen testing is the procurement of an amount of specimen adequate for proper isolation and/or identification of the analyte in question. Unfortunately, most currently used respiratory specimen collection methods are fairly invasive and uncomfortable and frequently don't allow for adequate visualization or determination of the amount of specimen collected. In the case of swab specimen collection, the practitioner may fail to collect any specimen on the swab. This creates significant variability in the amount of specimen available for testing and can easily lead to false negative results and reduced sensitivity of many diagnostic tests, especially those requiring a specific quantity of analyte.

Current techniques for collecting respiratory specimens from the nose, nasopharynx, and pharynx include several methods that are usually performed in a medical setting by a person having special skills. The current methods for collecting nasal, nasopharyngeal, and pharyngeal specimens include the use of: 1) nasal swabs, 2) nasopharyngeal swabs, 3) nasal washes, 4) nasopharyngeal aspirates, and 5) pharyngeal swabs.

Nasal swabs typically have tips made of Dacron, rayon, or foam, and are approximately the size of a Q-tip. The nasal swab tip is securely attached to a shaft that is usually made from a material such as plastic. Respiratory specimens are collected using nasal swabs by introducing the tip of the swab through the nasal opening and into the nasal antrum in a primarily vertical plane. The swab is then gently rotated while rubbing it along the nasal mucosal surface in order to trap or absorb respiratory secretions on the swab tip. A specimen may be collected from one or both nostrils, using one or more swabs.

Nasopharyngeal swabs have a shaft and tip similar to those found on nasal swabs; however, the shaft and tip are usually of a smaller diameter in order to facilitate passage of the swab into the nasopharynx. This collection procedure requires insertion of the nasopharyngeal swab through the nasal opening in a plane that is horizontal to the bottom of the nose. The swab is gently inserted into the nasopharynx (approximately half of the distance from the nasal opening to the front of the ear lobe), and the specimen is collected by gently rotating the swab to trap or absorb specimen on the swab tip.

Nasal washes are performed by forcing sterile saline solution or water into one nasal opening with a nasal bulb or syringe. The fluid is then either collected in a vessel held below the opposite nasal opening or is suctioned back into the bulb syringe that introduced the fluid.

Nasal and nasopharyngeal aspirates are collected via small flexible tubing connected to a vacuum source. The tubing is inserted into a single nasal opening and threaded into the nasopharynx. The vacuum suctions the nasal secretions into a collection container. Small amounts of sterile saline may be instilled into one or both nares to facilitate flow of the specimen through the tubing.

Pharyngeal swabs are similar to nasal swabs in size and configuration. Pharyngeal swabs are inserted over the back of the tongue and rubbed against the tonsils, uvula, and posterior pharynx.

All of the above collection techniques are invasive in that they involve insertion of foreign objects into bodily openings sufficient to inflict mild to moderate discomfort on patients, particularly children. The nasal and nasopharyngeal (NP) sampling methods include the potential to cause a nosebleed through introduction of the sampling device into the nose or nasopharynx. Pharyngeal (throat) swabs are uncomfortable and frequently cause the patient to gag or vomit due to stimulation of the pharyngeal tissue.

Non-invasive respiratory specimen collection refers to methods of collection that do not involve the insertion of an object into the patient's nose, mouth, or pharynx. Non-invasive collection methods can generally be performed by a single individual without special medical training, however in the case of small children and infants, minimal assistance may be required.

In a study presented by Bonner, et. al in 2007, patients presenting with acute respiratory illness were recruited to provide a respiratory specimen collected by one of the conventional methods (NP aspirate, NP swab, or nasal swab) and a second “blow nose” sample collected by blowing into a tissue-like material. Each sample was divided equally in order to perform rapid testing and viral culture for comparison.

A total of 487 patients provided both conventional and “blow nose” respiratory specimens for rapid testing and viral culture. Patients ranged in age from 16 months to 86 years and consisted of 42.9% males and 57.1% females. Study personnel documented that 409 (84%) patients provided a “blow nose” specimen that appeared adequate for testing. The respiratory samples obtained by having a patient blow his nose into a tissue-like material had rapid influenza test performance characteristics that were comparable to those of conventional respiratory samples using viral culture as the gold standard.

In a 2008 study by Bonner, et. al, samples of “blow nose” nasal secretion and cough droplets combined proved more efficacious for influenza diagnosis than combined nose and throat swab samples. The present invention, using “blow nose” and other non-invasively collected samples, provides an easy, non-invasive method of performing rapid influenza testing.

In U.S. Pat. No. 6,811,971, Klepp claims a method for diagnosing influenza A or B with tests performed on saliva samples. Klepp describes embodiments of his claimed method that cannot be characterized as “rapid” because of involved sample preparation and treatment Although stating that “a rapid individual diagnosis would be helpful as a basis for treatment decisions,” (column 2, line 20), Klepp describes only one embodiment as “rapid.” However, Klepp's sole rapid test “does not differentiate between influenza A and influenza B viruses” (column 8, line 31).

In U.S. Pat. No. 6,991,898, O'Conner claims an invasive diagnostic test method. The method is limited to the use of a swab for sample collection.

In US patent publication 2003/0120180, Kaylor et al. claim an invasive diagnostic test method. The method is limited to sample collection by means of a gloved finger.

In U.S. Pat. Nos. 6,551,791, 6,951,730, and 7,270,974, Small et. al claim a rapid, non-invasive diagnostic method of testing nasal secretions and “simply distinguishing between allergies and infections” ('791 column 8, line 62; '730 column 8, line 66; '974 column 8, line 13). The Small methods do not specifically differentiate among the many types of viral pathogens, including but not limited to influenza A and influenza B. “Viral infections can be deduced by the absence of allergy or sinusitis” ('791 column 7, line 15).

### BRIEF DESCRIPTION OF THE DRAWINGS

FIG. 1 illustrates a preferred embodiment of a non-invasive collection device that may be used in the present invention method.

FIG. 2 illustrates an alternate embodiment of a non-invasive collection device that may be used in the present invention method.

### DETAILED DESCRIPTION OF THE PREFERRED EMBODIMENT

Definitions. Terminology used herein describes particular embodiments only, and is not intended to be limiting. As used in the specification, including the claims, the singular forms “a”, “an”, and “the” include singular and plural referents unless the content dictates otherwise. For example, reference to “a sample” includes one or more samples. Unless defined otherwise, all technical and scientific terms used herein have meanings commonly understood by one of ordinary skill in the relevant art or industry.

“Analyte” herein generally refers to a chemical or biological agent. Biological agents include, but are not limited to, viruses, diseases, molds, allergens, and living organisms. One example of a chemical agent is lead.

“Being” herein includes humans and animals.

“Direct fluorescent antibody” (DFA) herein generally refers to a technique intended to detect the presence of a target analyte in a sample through use of antibodies tagged with fluorescent dye. This technique offers straightforward detection of antigens using fluorescently labeled antigen-specific antibodies.

“Director” herein generally refers to any device designed to direct, divert, or otherwise aim a sample in a desired direction.

“Indicator” herein generally refers to a solid, liquid, or gas having a characteristic that, in the presence of an entity that is the object of detection or measurement, changes sufficiently to show the presence of and/or the quantity and/or quality of such subject.

“Lateral flow test” herein generally refers to a technique intended to detect the presence (sandwich assay) or absence (competitive assay) of a target analyte in a sample. Also known as lateral flow immunochromatographic assays, these tests are often produced in a dipstick format, but can be used in any orientation. Lateral flow tests are a form of immunoassay in which a sample flows along a solid substrate via capillary action. After the sample is applied to the test it encounters a reagent that mixes with the sample and transits the substrate encountering lines or zones that have been pretreated with an antibody or antigen. Depending upon the analytes present in the sample, the reagent can become bound at the test line or zone.

“Molecular test” herein generally refers to a technique intended to detect the presence of a target analyte in a sample though detection of viral DNA that is pathogen-specific.

“Non-invasive” herein generally refers to a device or technique that requires minimal or no insertion into an opening, and that causes no significant discomfort to the patient.

“Opening” herein generally refers to a point of ingress or egress on an object, or, in the case of a human or other animal, an orifice such as the nose or an opening such as a wound.

“Proximal” and “distal” herein generally refer, respectively, to a point positioned nearest to and furthest from a point of reference. The point of reference herein is the opening of a subject from whence a biological sample is collected.

“Rapid” herein generally refers to a time span of less than one hour.

“Sample” or “biological sample” herein refers to a substance or combination of substances that can be tested for the presence or concentration of a particular component. Examples of samples that may be tested using the present invention include, but are not limited to blood, serum, plasma, saliva, sputum, urine, ocular fluid, tears, semen, vaginal discharge, nasal secretions and droplets, ear secretions, perspiration, mucus, stool, and amniotic, spinal, wound, or abscess fluid, sewage, milk, wine, and water from oceans, lakes, rivers, homes, factories, and runoff. Viscous fluids and semi-solid or solid specimens, for example plaque, skin, scalp, or paint scrapings, may be used to create liquid solutions, eluates, suspensions, or extracts that may serve as samples. Samples may include any combination of liquids, solids, and gases, and any biological materials including, but not limited to, cells, microbes, organelles, mold, and biochemical complexes. Samples are not limited to a single component. A sample may be, for example, a mixture of nasal secretion and cough droplets. A sample may include additives added for any purpose, for example, a catalyst.

The present invention eliminates or alleviates the above-mentioned problems and limitations of state of the art diagnosis methods. The present invention provides a method for rapid diagnosis of specific pathogens in a sample collected non-invasively.

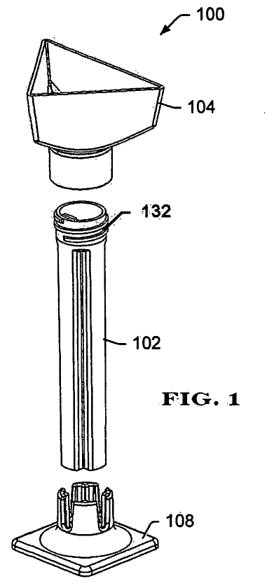

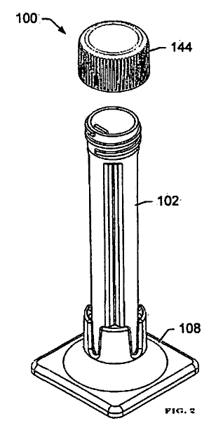

One embodiment of the present invention is a method of making medical diagnoses using non-invasive sample collection tools such as those described by Bonner in U.S. patent application Ser. No. 12/006,662 (publication US 2009/0038416 A1), which is hereby incorporated by reference. For nasal mucus, droplets from coughing or sneezing, saliva, and pharyngeal and oropharyngeal fluid, one embodiment of the present invention method consists of testing such effluent collected by urging the patient to blow his or her nose, or spit, dribble, cough, or sneeze into a collection device such as that illustrated in FIGS. 1 and 2.

FIGS. 1 and 2 depict a preferred embodiment of one non-invasive collection device that may be used with the present invention method for testing samples. In some embodiments, device 100 for collecting samples and investigating the presence of biological or chemical entities may include a sample receptacle 102, a sample director 104, and at least one indicator (not shown) that may be positioned inside receptacle 102 before or after a sample is collected. A collection device may include a receptacle base 108 and/or a plug or cap 144 and receptacle threads 132 that cooperate to contain the sample in the receptacle. A collection device may include a director with any shape convenient for sample collection, for example a director shaped to conform to a particular body opening or orifice. A collection device director or receptacle may include provision for overflow or blow-by, for example when a sample is expectorated into a director with a forceful stream of air.

In the present invention, a sample collected in a non-invasive collection device is brought in contact with an indicator capable of producing a signal, in situ or subsequent to some secondary event, that indicates the presence or absence, quantity, or quality of an entity that is the subject of analysis. The indicator may be a lateral flow test, direct fluorescent antibody test, or a molecular-based test method.

Some indicators are intended to provide purely qualitative results. However it is possible to measure the intensity of a test line to determine the quantity of analyte in a sample. Implementing a magnetic immunoassay in a lateral flow test also provides a quantified result. While not strictly necessary, many lateral flow tests incorporate a second line (control) that contains an antibody the purpose of which is confirmation of test accuracy. Time required to obtain the test result is a key feature of these tests, some of which require only a few minutes. Generally there is a trade off between time and sensitivity; more sensitive tests may take longer to develop. The other key advantage of lateral flow testing compared to other immunoassays is simplicity; the tests typically require little or no sample or reagent preparation.

Examples of biologic agent detection by commercially available rapid indicators:

Adenovirus

DHI Adenovirus DFA Reagent: Product Code 01-013402.v2

SAS Adeno Test: Product Code 067020

Influenza A and/or B

3M™ Rapid Detection Flu A+B Test

Clearview Exact Influenza A & B: Product Code 506761

DHI Influenza A DFA Reagent (2-mL): Product Code 01-013102.v2

DHI Influenza B DFA Reagent (2-mL): Product Code 01-013202.v2

Directigen Flu A (Becton-Dickinson): Product Code 256020

Directigen EZ Flu A+B (Becton-Dickinson): Product Code 256050

BinaxNOW Influenza A&B (Inverness): Product Code B416-000

OSOM® Influenza A&B (Genzyme)

QuickVue Influenza Test (Quidel): Product Code 00317

QuickVue Influenza A+B Test (Quidel): Product Code 20183

SAS FluAlert (SA Scientific)

TRU FLU (Meridian Bioscience)

XPECT Flu A&B (Remel)

Human Metapneumovirus

DHI Metapneumovirus DFA Reagent (5-mL): Product Code 01-035005

Parainfluenza

DHI Parainfluenza 1 DFA Reagent (2-mL) 01-013502.v2

DHI Parainfluenza 2 DFA Reagent (2-mL) 01-013602.v2

DHI Parainfluenza 3 DFA Reagent (2-mL) 01-013702.v2

Respiratory Syncytial Virus

3M Rapid Detection RSV Test

BinaxNOW RSV Test Kit: Product Code B430-022

ClearView RSV Rapid Test Kit: Product Code 135060

QuickVue RSV Test: Product Code 20193

SAS RSV Alert Testing Kit: Product Code 041130

TRU RSV (Meridian Bioscience)

I claim:

1. A method for rapid determination of the presence or absence of a specific biological entity in a being, said method comprising the steps of:
causing the contact of an indicator with a sample, said sample having been taken from said being using a non-invasive collection device; and
observing subsequent reaction of said indicator.

2. A method for rapid determination of the presence or absence of a specific viral pathogen in a being, said method comprising the steps of:
causing the contact of an indicator with a sample, said sample having been taken from said being using a non-invasive collection device; and
observing subsequent reaction of said indicator.

3. A method for rapid determination of the presence or absence of a specific chemical entity in a being, said method comprising the steps of:
causing the contact of an indicator with a sample, said sample having been taken from said being using a non-invasive collection device; and
observing subsequent reaction of said indicator.

4. A method for rapid determination of the specific presence or absence of influenza A in a being, said method comprising the steps of:
causing the contact of an indicator with a sample, said sample having been taken from said being using a non-invasive collection device; and
observing subsequent reaction of said indicator.

5. A method for rapid determination of the specific presence or absence of influenza B in a being, said method comprising the steps of:
causing the contact of an indicator with a sample, said sample having been taken from said being using a non-invasive collection device; and
observing subsequent reaction of said indicator.

6. A method for rapid determination of the specific presence or absence of respiratory syncytial virus in a being, said method comprising the steps of:
causing the contact of an indicator with a sample, said sample having been taken from said being using a non-invasive collection device; and
observing subsequent reaction of said indicator.

7. A method for rapid determination of the specific presence or absence of adenovirus in a being, said method comprising the steps of:
causing the contact of an indicator with a sample, said sample having been taken from said being using a non-invasive collection device; and
observing subsequent reaction of said indicator.

8. A method for rapid determination of the specific presence or absence of parainfluenza in a being, said method comprising the steps of:
causing the contact of an indicator with a sample, said sample having been taken from said being using a non-invasive collection device; and
observing subsequent reaction of said indicator.

9. A method for rapid determination of the specific presence or absence of metapneumovirus in a being, said method comprising the steps of:
causing the contact of an indicator with a sample, said sample having been taken from said being using a non-invasive collection device; and
observing subsequent reaction of said indicator.

10. A method for rapid determination of the specific presence or absence of a subtype of influenza A in a being, said method comprising the steps of:
causing the contact of an indicator with a sample, said sample having been taken from said being using a non-invasive collection device; and
observing subsequent reaction of said indicator.

11. A method for rapid determination of the specific presence or absence of an adenovirus subtype in a being, said method comprising the steps of:
causing the contact of an indicator with a sample, said sample having been taken from said being using a non-invasive collection device; and
observing subsequent reaction of said indicator.

12. A method for rapid determination of the specific presence or absence of respiratory syncytial virus type in a being, said method comprising the steps of:
causing the contact of an indicator with a sample, said sample having been taken from said being using a non-invasive collection device; and
observing subsequent reaction of said indicator.

13. A method for rapid determination of the specific presence or absence of parainfluenza type in a being, said method comprising the steps of:
causing the contact of an indicator with a sample, said sample having been taken from said being using a non-invasive collection device; and
observing subsequent reaction of said indicator.
